(19) United States
(12) Patent Application Publication
(10) Pub. No.: US 2014/0178311 A1
(43) Pub. Date: Jun. 26, 2014

(54) METHOD OF ADDING BOTANICAL AGENTS/DIETARY SUPPLEMENTS TO PHARMACEUTICAL AGENTS IN A PHARMACOTHERAPEUTIC REGIMEN

(71) Applicants: Joshua D. Levine, Chapel Hill, NC (US); Robert A. Levine, Guilford, CT (US)

(72) Inventors: Joshua D. Levine, Chapel Hill, NC (US); Robert A. Levine, Guilford, CT (US)

(21) Appl. No.: 14/062,518

(22) Filed: Oct. 24, 2013

Related U.S. Application Data
(63) Continuation of application No. 13/146,835, filed on Aug. 25, 2011, now abandoned.
(60) Provisional application No. 61/148,265, filed on Jan. 29, 2009.

(51) Int. Cl. A61K 36/68; A61K 35/74; A61K 9/28; A61K 36/288; A61K 36/185; A61K 9/48; A61K 31/451; A61K 31/192

(52) U.S. Cl. CPC A61K 36/68; A61K 31/451; A61K 35/747; A61K 31/192; A61K 36/288; A61K 36/185; A61K 9/4891; A61K 9/288
USPC 424/10.2; 424/738; 424/93.45; 424/764; 424/725; 424/463; 424/475

(57) ABSTRACT

A method for administering an active pharmaceutical agent (APA) is provided that includes the steps of: a) providing a medicine having a dose of at least one APA and at least one active botanical agent (ABA) or dietary supplement; and b) dosing the APA within the medicine such that within each subsequent dose of the medicine, the amount of the APA decreases and the amount of the ABA increases, or remains the same, or decreases relative to an earlier amount. A trace of the APA may or may not remain in the pill toward the end of medicine application.

This patent application is a continuation of U.S. patent application Ser. No. 13/146,835 filed Aug. 25, 2011, which claims priority from PCT Patent Application No. PCT/US2010/022394 filed Jan. 28, 2010, which claims priority to U.S. Provisional Application No. 61/148,265 filed Jan. 29, 2009, all of which are hereby incorporated by reference in their entireties.

### BACKGROUND OF THE INVENTION

1. Technical Field

This invention relates to methods for administering an active pharmaceutical agent (APA) in general, and to methods and agents that improve the aforesaid administration in particular.

2. Background Information

Plant medicines have been employed for thousands of years to effectively treat a multitude of illnesses and health conditions. Starting in the 1940s, chemically synthesized pharmaceuticals began replacing herbal medicines in the U.S. Approximately three-quarters of all commonly used medications are now wholly chemically synthesized pharmaceuticals, while the remaining 25% of pharmaceuticals contain some compound derived from plants. Most of these pharmaceuticals in the remaining 25%, while having components derived from plant sources, are also highly refined and/or chemically synthesized. Medicines containing plant material combined with chemically defined active substances, including chemically defined, isolated constituents of plants, are not considered to be herbal medicines.

These synthesized pharmaceuticals, while offering great benefits in many medical conditions, are not without shortcomings. They are often expensive to use, and frequently have high levels of dose related toxicity. Many pharmaceuticals also often produce unwanted side effects which while not toxic or very dangerous, will significantly decrease the likelihood that the patient will comply with the prescribed regimen. While there are also plant medicines that contain high levels of toxicity and produce unwanted side effects, there are also many plant medicines that have very low levels of toxicity, and are generally recognized as safe (i.e., “GRAS”). They have been shown in verifiable scientific studies to be effective in treating various ailments. It is this class of GRAS plant medicines (and other dietary supplements) that may be added to conventional pharmaceutical drugs (once any contraindications have been ruled out) to accomplish the goals of the present invention.

Plant medicines (also known as herbal medicines or botanical medicines) contain parts of plants as active ingredients, whether in the crude state or as plant preparations. Plant materials may include extracts, plant juices, gums, fatty oils, and other substances of this nature. An ABA (active botanical agent) is an herbal medicine that includes refined or unrefined extracts of whole or parts of plants, or botanicals in crude state.

The term “therapeutic effect” as used herein is defined as an effect that causes the patient to feel better or heal by ameliorating the signs and/or symptoms of the disorder, disease, or side effects of an APA or any combination thereof.

The term “pharmacologic action” is defined herein as the biochemical or physical mechanism of an active pharmaceutical agent's (APA's) or ABA's activity, both desirable and undesirable, if known. For example, three drugs that treat constipation all are said to have the same “therapeutic effect”—they increase the likelihood of defecation. They may each have a different pharmacologic action. Psyllium seed husks act as laxatives by drawing water into the colon. Phenolphthalein is a laxative that stimulates the intestinal motility and is said to be a stimulant laxative. Docusate is a laxative that is a detergent and works as a stool softener. One could add psyllium seed husks to a laxative drug that has a different pharmacologic action and the therapeutic effects would be complimentary. One could add psyllium seed husks to a drug such as morphine that causes constipation as a side effect, and the therapeutic effects would counteract the side effects of the drug.

### SUMMARY OF THE INVENTION

It is an object of this invention to combine one or more active pharmaceutical agents (APAs) with one or more active botanical agents (ABAs) or dietary supplements in the same pill, with the ABAs including constituents that are formulated to achieve at least one of the following goals: enhance the actions of the APA (with similar acting actions); complement the actions of the APA (with different, but complementary actions); and/or counteract one or more of the side effects of the APA. As the total amount of APAs decreases and is replaced with ABAs in the pharmacotherapeutic regimen, deleterious effects caused by the APA will also decrease. Many side effects of APA are dose related. The present invention accomplishes reduction of these side effects by allowing reduction in the amount of APA per dose of medication and/or by using ABA that either counteracts the side effects and/or permits the lowering of the APA amount per dose of medication by having an additive salutary effect similar to the desired effect of the APA.

It is an object of the present invention to provide an administration method that causes a decrease in potential side effects from administered pharmaceuticals. It is another object of the present invention to provide an ABA that complements the therapeutic effects of the APA. Another object of the present invention is to provide a method that allows the APAs in a pharmacotherapeutic regimen to be replaced with substances that are generally recognized as safe (GRAS substances). Another object of the present invention is to provide a patient with one pill or one set of pills that contain both complementary medicines (ABA and APA) in the prescribed amounts, thereby reducing patient dosing errors, and increasing patient compliance.

The present invention includes a pharmaceutical medicine. In more specific terms, the present invention involves the use of an active botanical agent (ABA) or other dietary supplement in combination with an active pharmaceutical agent (APA) in order to accomplish one or more salutary effects. The present invention includes methods and medicines that are operative to decrease the dose of active pharmaceuticals (chemically synthesized drugs) administered to a patient during a course of treatment while maintaining a high level of efficacy and improving patient compliance.

According to an aspect of the present invention, a method for administering an active pharmaceutical agent (APA) is provided that includes the steps of: a) providing a medicine having a dose of at least one APA and at least one active botanical agent (ABA) or dietary supplement; and b) dosing the APA within the medicine such that within each subsequent dose of the medicine, the amount of the APA decreases and the amount of the ABA increases or remains the same as an earlier amount. A trace of the APA may or may not remain in the pill toward the end of medicine application.

According to another aspect of the present invention, a pharmaceutical medicine is provided that includes: a) a dose of at least one APA; and b) an amount of at least one ABA or a dietary supplement, wherein the APA and the ABA exist in a stable relationship throughout the course of treatment.

### DETAILED DESCRIPTION OF THE INVENTION

The present invention includes a method for administering one or more APAs to a subject, and a medicine containing one or more such APAs. The medicine includes one or more ABAs, and may assume a variety of different forms (e.g., pill, capsule, liquid, etc.), and is not limited to any particular form. In some embodiments, the ABA may be incorporated into the medicine as a coating on a pill or capsule, or as part of the capsule itself. When the medication is in a simple liquid form, or within a more complex delivery system such as a suspension, hydrosol, dermatological patch, ointment, gel and/or a cream, etc., either the APA or the ABA, or both, may be in the liquid, solution, suspension, or gel. A capsule may contain either solid or liquid forms of the APA and/or the ABA. The capsule may be coated with an ABA, or may include an ABA within the material of the capsule.

The constituents within the medicine can be selected so that the ABA enhances (with similar pharmacologic actions) or compliments (with different pharmacologic actions) the APA, and both agents may stay in a stable ratio (i.e., a stable relationship), or the amount of APA may decrease as the amount of ABA increases. The constituents within the medicine can be selected so that the ABA counteracts side effects caused by the APA, and the amount of ABA may remain constant, or may diminish or increase as the dose of APA is decreased.

The present method for administering the medicine includes embodiments wherein the relative amounts of the ABA and APA within the medicine vary periodically over the course of the regimen.

The duration and type of pharmaceutical therapy prescribed by a physician or health care provider to a patient is dependent upon the physician's judgment about nature of the illness and the patients response to therapy. Optimal medical care requires that the doctor-patient relationship be maintained and that professional judgment (aided by patient examination and/or interaction) be used to determine the appropriate response to therapy. For example if under the present invention an ABA is administered to a patient to decrease the side effects of an APA, then over the course of therapy the dosage of the ABA could be maintained constant or decreased as the dosage of the APA was decreased (e.g., pursuant to a physician's care). In this manner, the side effects associated with the APA could be mitigated with an appropriate amount of an ABA. Alternatively, if an ABA was administered to enhance the therapeutic effects of an APA under the present invention, then over the course of therapy the dosage of the ABA could be maintained at or decreased from the earlier ABA dosages, while at the same time the dosage of the APA would be decreased (e.g., pursuant to a physician's care). In this manner, the side effects associated with the APA could be mitigated with an appropriate amount of an ABA. As a further example, in instances where the physician wants to wean a patient off a medication, then under the present invention both the APA and ABA could be decreased over the course of the therapy; e.g., decreased in manner where the APA and ABA are maintained in a stable relationship (relative amounts remaining substantially the same), while the amount of medication is decreased over time. As a still further example, in instances where the patient has a chronic condition, the attending physician could under the present invention elect to keep the dosage of the APA and ABA constant over the course of therapy.

Under the present invention, the ABA may be added to an existing brand-name or generic pharmaceutical, or an entirely new drug may be formulated consisting of both ABA and APA constituents in the same pill.

Herbal medicines or plants extracts are currently classified by the FDA not as drugs, but as dietary supplements. This broad category of dietary supplements also includes vitamins, minerals, enzymes, beneficial bacteria, and other nutritional products. This invention seeks to cover the synchronized use (as described) of pharmaceuticals and any plant medicine or dietary supplement where the combination of ingredients is prepackaged in a single dose form.

The prescribed combinations of APA and ABA may be formulated by the manufacturer to provide the physician with choices in the manner of administering the prescribed regimen, and can be presented to the patient in a blister-type pack, or other sequential delivery systems such as is employed in oral contraceptive delivery systems, with each day's dose clearly indicated either directly on the pill and/or on the packaging containing the pill(s). The pill(s) for a given day can be marked with their day and/or sequence number. In a preferred Bann of the invention, the patient will be taking the same number of identical appearing pills each day, thereby greatly increasing patient compliance and decreasing dosage errors.

According to one aspect of the invention, the method includes the steps of: a) providing a medicine having a dose of at least one APA and at least one active botanical agent (ABA) or dietary supplement; and b) administering the medicine in an arranged regimen so that subsequent doses of the medicine include a decreasing amount of at least one APA. In some embodiments, the amount of the ABA (or at least one of the ABAs) within the regimen of medicine increases as the regimen progresses. In other embodiments, the amount of the ABA (or at least one of the ABAs) within the regimen of medicine decreases as the regimen progresses. In still other embodiments of the regimen of the medicine, the amount of the ABA within the regimen of medicine remains constant as the regimen progresses. At the end of the regimen, the amount of APA within the medicine may be a trace amount (e.g., a level below that which is generally recognized as therapeutically active, herein known as inactive) or none at all.

According to another aspect of the invention, a pharmaceutical medicine is provided that includes a dose of at least one APA, and an amount of at least one ABA.

In some embodiments, the ABA within the medicine includes one or more constituents that have the same or similar therapeutic effect as the therapeutic effect of the APA within the medicine, thereby permitting the total dose of the APA to be decreased, and lessening the total amount of toxicity and dose related side effects caused by the APA.

In some embodiments, the ABA includes one or more constituents that have therapeutic effect that is complimentary with the therapeutic effect of the APA. The constituents within the ABA may provide the therapeutic effect in a manner different from therapeutic effect provided by the APA, thereby potentially decreasing side effects caused by the APA by lowering the total dose of APA ingested.

In some embodiments, the ABA includes one or more constituents that are counteractive to one or more side effects caused by the APA, and thereby enables better tolerance of an efficacious dose of an APA.

In some embodiments, amounts of medicine administered within the regimen may be packaged in a set sequence (e.g., a blister pack with numbers or days of the week indicating the order the pills are to be taken) that dictates the order they are to be taken.

In some embodiments, the amounts of APA and ABA within the medicine administered may be staggered. For example, a full dose of APA may be delivered one day (with little to no ABA). On the next day a full dose of ABA may be delivered one day (with little to no APA). This could effectively cut the dose of administered APA by fifty percent. Other spacings would result in different dose administrations.

According to another aspect of the present invention, a method for administering an APA is provided that includes the steps of a) providing a first day's dose schedule of medicine having a full effective amount of at least one APA and at least one ABA; and b) providing a subsequent day's dose schedule of medicine having a relatively low amount of at least one APA and at least one active botanical agent (ABA) or dietary supplement and continuing the first and subsequent day's dose schedule for a predetermined period of time. There may be decrements in APA on subsequent days. In some embodiments of this aspect, the effective amount of the APA can be decreased over the regimen. In those embodiments, the effective amount of the ABA may increase where the amount of the APA decreases.

According to another aspect of the invention, a method for administering an APA is provided that includes the steps of a) providing a prepackaged set of sequential doses (e.g., blister pack with number of days of the week indicating the order the doses are to be taken); and b) administering a dose of an ABA in one of the following manners, or some combination/variation thereof: 1) prior to treatment with an APA; 2) during the course of treatment with an APA; and 3) at the end of a treatment with an APA.

In those instances where the doses are in pill or capsule form, the pills or capsules may be identical in form throughout the regimen. The amount of ABA and/or APA within each dose may vary during the course of the regimen.

### EXAMPLES

In some cases the goal of the pharmacotherapeutic regimen of this invention is to substantially or completely replace the APA with a similarly acting ABA. For example in the case of a patient with irritable bowl syndrome (IBS), the patient may be taking antidiarrheal agents such as Loperamide. Side effects of this APA include allergic reactions, abdominal distention, constipation, drowsiness, and dry mouth. This is particularly unfortunate as constipation is also a major symptom of IBS. Psyllium seed (Plantago ovata) may be added to these APAs which would: 1) help alleviate any possible constipation that may occur with taking the pharmaceuticals; and 2) could gradually replace the pharmaceutical agents in the pills given during the course of treatment. Psyllium seed has been used by herbalists and medical doctors alike to regularize bowel movements, and has no known side effects.

In other cases the goal of the regimen is to counteract side effects of the pharmaceuticals. For example, one serious side effect that often occurs with a patient taking a course of antibiotics is that the beneficial bacteria are destroyed along with the harmful ones. In this case, the antibiotic regimen may contain, at some point during treatment, a dose of Lactobacillus acidophilus and/or other beneficial bacteria. This dose of restorative beneficial bacteria may be given concurrently in the same pill with each dose of the antibiotic, or may occur in the last pills (set in a blister pack) given in the treatment regimen.

Another example where the goal of the regimen is to counteract the side effects of a pharmaceutical might occur when a patient is prescribed ibuprofen (or some other non-steroidal anti-inflammatory drug; i.e., “NSAID”) for pain or for an inflammatory condition such as arthritis. NSAIDs can cause salt-retention leading to weight gain due to edema, and sometimes hypertension. This problem may be addressed by adding an extract of dandelion leaf (Taraxacum officinal), which is a mild diuretic, to the NSAID. This may decrease the incidence of weight and hypertension which is associated with NSAID use.

In some cases the goal of the regimen is for the ABA to complement the actions of the APA. Various benzodiazepines are often used to treat insomnia. Side effects include clumsiness, drowsiness, dizziness, or may be habit forming or cause temporary impairment of short-term memory. Extracts of passionflower (Passiflora incarnata) have been shown to be highly effective in cases where an overactive mind or worry is interfering with the sleep cycle. The extract could be combined in a pill with a low dose of a benzodiazepine for patients experiencing insomnia from an overactive mind. The dose of pharmaceutical may be decreased as the dose of Passiflora extract is maintained or increased.

Patients most likely to benefit by the present invention are those taking APAs for the signs or symptoms associated with acne, altitude sickness, angina, anxiety, arthritis, asthma, bladder infections, body odor, breast cysts, breast pain, breast-feeding problems, bronchitis, bursitis, canker sores, cardiac arrhythmia, carpel tunnel syndrome, cold sores, colds and flu, constipation, depression, diabetes, diarrhea, diverticulosis, ear infections, eating disorders, eczema, endometriosis, fatigue, fibroids, flatulence, fungal skin infections, gallstones, glaucoma, gout, gum disease, hangover, hay fever, headaches, heart disease, heart burn, hemorrhoids, herpes, high blood pressure, high cholesterol, hives, hypothyroidism, indigestion, insomnia, intermittent claudication, intestinal parasites, irritable bowel syndrome, libido problems, memory loss, obesity, menopause, menstrual problems, motion sickness, multiple sclerosis, nausea, nerve pain, obsessive compulsive disorder, osteoporosis, Parkinson's disease, pneumonia, poison ivy, prostate enlargement, Raynaud's phenomenon, sinus infections, smoking addiction, sore throat, stress, stroke, toothache, and ulcers.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed herein as the best mode contemplated for carrying out this invention.

What is claimed is:

1. A method for administering a prescribed medicine that includes an active pharmaceutical agent (APA), comprising the steps of:
providing a an orally administered prescribed medicine having a dose of at least one APA and at least one active botanical agent (ABA);
dosing the APA within the medicine such that within subsequent doses of the medicine, the dose of the APA decreases and the dose of the ABA increases, decreases or remains the same as an earlier amount; and
administering the medicine in sequential order having a last application of the medicine, and wherein the dose of the APA in the last application is below the starting dose.

2. The method of claim 1, wherein the ABA includes one or more constituents that have a therapeutic effect that is substantially the same as a therapeutic effect provided by the APA.

3. The method of claim 1, wherein the ABA includes one or more constituents that have one or more therapeutic effects that are complimentary with a therapeutic effect of the APA.

4. The method of claim 1, wherein the ABA contains one or more constituents that provide one or more pharmacologic actions that are different than pharmacologic actions provided by the APA to improve the therapeutic effect.

5. The method of claim 1, wherein the ABA includes one or more constituents that have a therapeutic effect that is counteractive to one or more side effects of the APA.

6. The method of claim 1, wherein the medicine is administered in a pill, tablet, or capsule form, which form is individually labeled with a visible indicia on its surface indicating the sequential order in which it is to be taken.

7. A method for administering a prescribed medicine that includes an active pharmaceutical agent (APA), comprising the steps of:
providing a an orally administered prescribed medicine having a dose of at least one APA and at least one active botanical agent (ABA); and
dosing the APA within the medicine such that within subsequent doses of the medicine, the amount of the APA and the amount of the ABA decrease;
administering the medicine in sequential order having a last application of the medicine, and wherein the dose of the APA in the last application is below the starting dose.

8. The method of claim 7, wherein the ABA includes one or more constituents that cause a physiological response that is the same or similar to the physiological response caused by the APA, thereby decreasing the total dose of the APA ingested, and lessening the total amount of toxicity and side effects caused by the APA.

9. The method of claim 7, wherein the ABA includes one or more constituents that are complimentary with the healing actions of the APA; and
wherein the ABA contains constituents that through different pharmacologic actions may heal the patient's illness in a manner different from pharmacologic actions of the APA, thereby potentially decreasing side effects caused by the APA by lowering the total dose of APA ingested.

10. The method of claim 7, wherein the ABA includes one or more constituents that are counteractive to one or more side effects of the APA, and thereby enable better tolerance of an efficacious dose of an APA.

11. The method of claim 7, wherein the medicine is in pill, tablet, or capsule form, and each pill, tablet, or capsule is individually labeled with a visible indicia on its surface indicating the subsequent order in which it is to be taken within the medicine application.

12. A method for administering a prescribed medicine that includes an active pharmaceutical agent (APA), comprising the steps of:
providing a first day dose schedule of an orally administered prescribed medicine having a full effective amount of at least one APA, and an amount of at least one of an active botanical agent (ABA);
providing a second day dose schedule of an orally administered prescribed medicine having an amount of the at least one APA that is substantially less than the amount of APA contained within the first day schedule, and an amount of the at least one of the ABA; and
administering the first day and second day dose schedule repetitively for a predetermined number of days.

13. The method of claim 12, wherein the effective amount of APA periodically decreases.

14. The method of claim 13, wherein the amount of ABA increases in the applications where the amount of APA decreases.

15. The method of claim 12, wherein the amount of ABA includes one or more constituents that have pharmacologic actions that are the same as or similar to pharmacologic actions of the APA.

16. The method of claim 12, wherein the ABA contains constituents having pharmacologic actions that heal a patient's illness in manner different from pharmacologic actions of the APA.

17. The method of claim 12, wherein the ABA includes one or more constituents having therapeutic effects that are counteractive to one or more side effects of the APA.

18. The method of claim 12, wherein the amounts of medicine are administered from a package where they are disposed in a set sequence that dictates the order they are to be taken.

19. The method of claim 12, wherein the ABA in each day's dose schedule is mixed with the APA.

20. The method of claim 12, wherein the APA is in the form of a pill or a tablet, or is contained within a capsule, and the ABA is coated on the respective pill, tablet, or capsule.

21. The method of claim 20, wherein each pill, tablet, or capsule is individually labeled with a visible indicia on its surface indicating the sequential order in which it is to be taken.

22. A method for administering a pharmacotherapeutic regimen of a prescribed medicine, comprising the steps of:
providing a prepackaged set of sequential orally administered prescribed pills, wherein each pill contains at least one active botanical agent (ABA), and at least one active pharmaceutical agent (APA), which APA and ABA have complimentary pharmacologic actions, and wherein all the pills are substantially identical looking; and
administering the pills in sequential order;
wherein the dose of at least one of the ABA or APA in each pill varies during the course of the regimen.

23. The method of claim 22, wherein the pills are individually labeled with visible indicia on a surface of the pill, indicating the sequential order in which the pills are to be taken.
